(19) United States
(12) Plant Patent Application Publication
(10) Pub. No.: US 2003/0226185 P1
(43) Pub. Date: Dec. 4, 2003

(54) Bienville sweetpotato

(76) Inventors: Don R. LaBonte, Baton Rouge, LA (US); James M. Cannon, Baton Rouge, LA (US); Christopher A. Clark, Baton Rouge, LA (US); Arthur Q. Villordon, Monroe, LA (US); Paul W. Wilson, Gonzales, LA (US); Abner M. Hammond, Baton Rouge, LA (US); Rick N. Story, Baton Rouge, LA (US)

Correspondence Address: PATENT DEPARTMENT, TAYLOR, PORTER, BROOKS & PHILLIPS, L.L.P, P.O. BOX 2471, BATON ROUGE, LA, 70821-2471, US

(21) Appl. No.: 10/158,425

(22) Filed: May 29, 2002

(51) Int. Cl. A01H 5/00

(52) U.S. Cl. PLT/258

(57) ABSTRACT

A new variety of sweet potato identified as “Bienville Sweetpotato” is disclosed having superior disease resistance to both southern root-knot nematode and soil rot, and high yield characteristics.

This invention pertains to a variety of sweetpotato having superior disease resistance to both southern root-knot nematode and soil-rot, and high yield characteristics.

This new variety is identified as “Bienville Sweetpotato” (“Bienville”). Bienville is a sweetpotato variety, Ipomoea batatas (L.) Lam., developed from a seedling produced in a polycross nursery. Bienville demonstrates superior southern root-knot nematode and soil-rot resistance, and high yield characteristics as compared to other available sweetpotato varieties. Bienville is characterized by a dark orange flesh and elliptical roots.

Sweetpotatoes, unlike Irish potatoes (Solanum tuberosum), are not tuber propagated plants. A “tuber” is a short, thickened portion of an underground branch. Along a tuber are found “eyes,” each of which comprises a ridge bearing a scale-like leaf (analogous to a branch leaf) having minute meristematic buds in the axial of the leaf. By contrast, sweetpotato roots are developmentally and anatomically true roots, lacking meristematic buds, and are not derived from an underground branch. Sweetpotatoes do not form tubers.

### BRIEF DESCRIPTION OF THE DRAWING

FIG. 1 is a color photograph of the fleshy root of the novel variety of sweetpotato identified as “Bienville.”

FIG. 2 is a color photograph of the fleshy root of the sweetpotato variety identified as “Beauregard.”

FIG. 3 is a color photograph of the canopy biomass of both the novel variety of sweetpotato identified as “Bienville” and the variety identified as “Beauregard.”

The file of this patent contains at least one photograph executed in color. Copies of this patent with color drawing(s) will be provided by the Patent and Trademark Office upon request and payment of the necessary fee.

Bienville, also identified by the seedling code L94-96, was developed at the Louisiana Agricultural Experiment Station in Baton Rouge, La., to provide a variety with characteristics generally similar to those of the Beauregard sweetpotato, but with improved resistance to southern root-knot nematode. Bienville originated in 1994 as a seedling from a polycross nursery of the previous year. The female parent of Bienville is L86-33. The female parent of L86-33 is Beauregard. The male parent of Bienville is unknown. All parents were derived from the sweetpotato breeding program at the Louisiana Agricultural Experiment Station.

Bienville roots were stored during the winter at the Louisiana Agricultural Experiment Station (Sweetpotato Research Station) in Chase, La. During the following spring, Bienville was planted and produced approximately 8- 10 sprouts, which were cut and transplanted successfully for asexual reproduction.

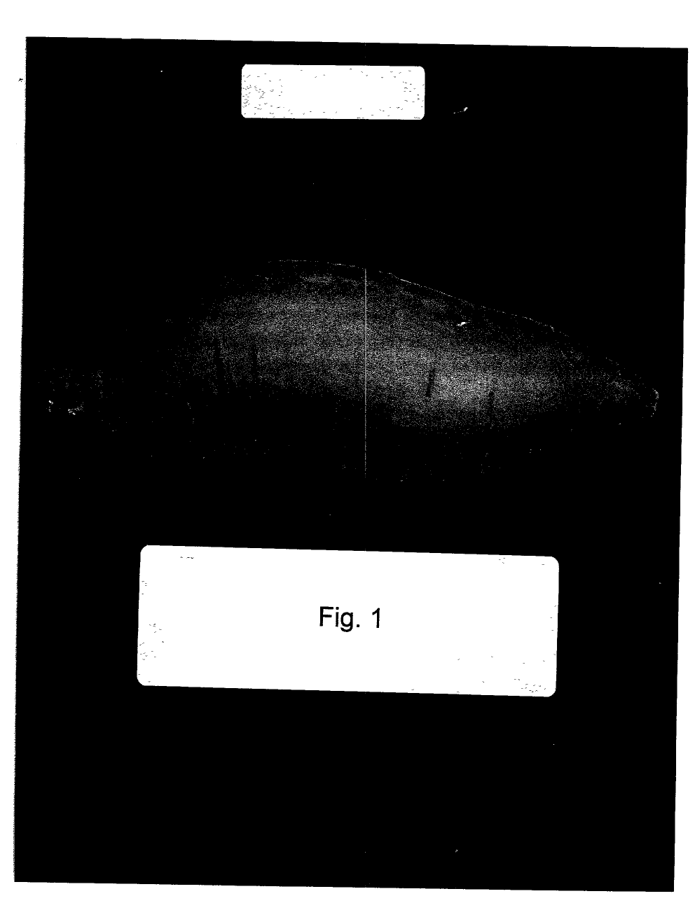

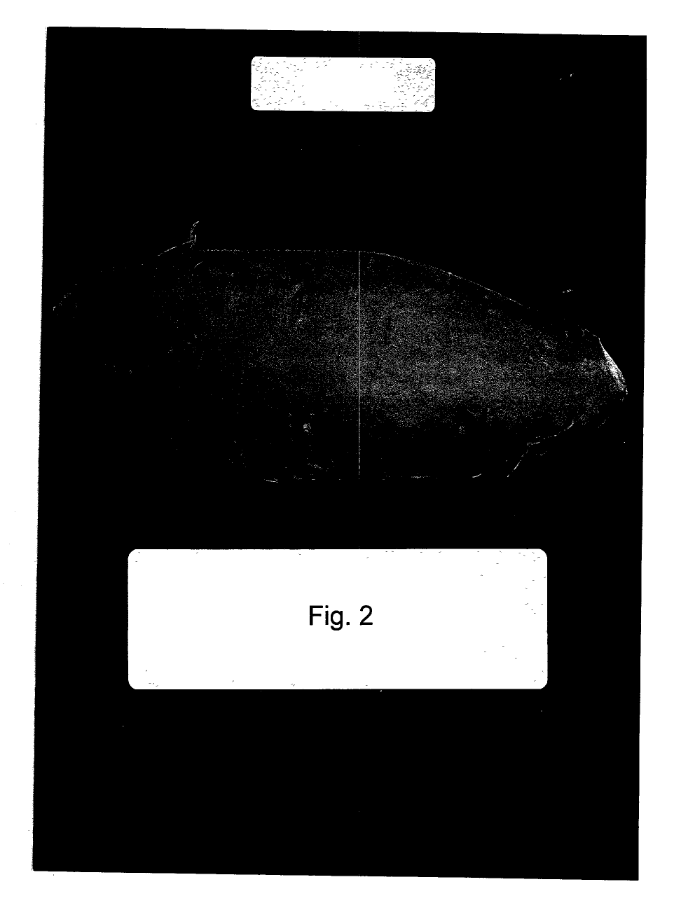

FIG. 1 depicts the fleshy root of the Bienville sweetpotato. Skin varies in color from light to medium rose, and is typically lighter than Beauregard at harvest. However, skin color is similar to Beauregard after several months of storage. (Color intensity fades in storage.) See Beauregard as depicted in FIG. 2 for comparison. Skin is smooth, and storage roots are elongate without lobing, similar to Beauregard. The cortex is 4-5 mm in depth.

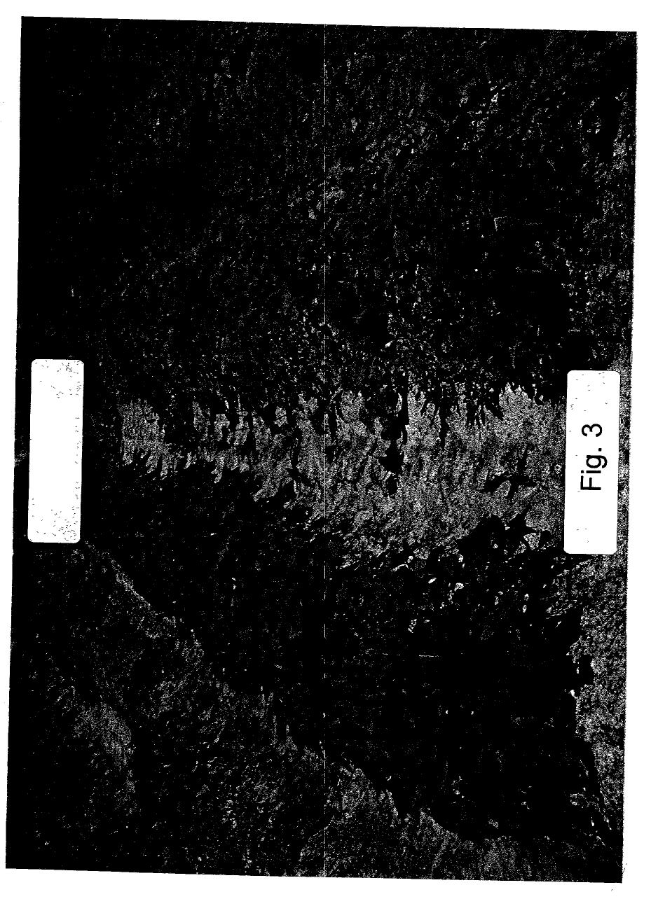

FIG. 3 depicts the canopy biomass of the varieties Bienville and Beauregard. Bienville's canopybiomass, located towards the left side of the photograph, has green-stemmed vines that extend from the apex to the crown of the roots. Bienville's canopy biomass appears much thicker than Beauregard's, shown on the right side of the photograph, but is only slightly thicker than that of Jewel (not shown). The canopy has an upright and erect architecture prior to spreading, unlike Beauregard's canopy, which has a prostrate growth habit. Unfolded immature leaves are dark purple with a few green abaxial veins. The immature leaves gradually change to dark green laminae at a distance of one to two nodes from the apex. A few vestigial purple mottlings are found on the third leaf from the apex. Mature leaves have an acute apex, and either a cordate or hastate base. Abaxial and adaxial veins are light purple. Leaves are generally darker in comparison to Beauregard. The petiole is purple at its junction with the leaf; however, it quickly changes to green, and then slightly purple at the node. The dormant nodal meristem is also purple.

Colorimetric evaluations using the Average Hunter Chromacity values (hue(L), value (a), and chroma (b)) of skin and flesh for both Bienville and Beauregard storage roots at harvest, are shown in Table 1, using conversion software provided by Munsell Color Services (New Windsor, N.Y.).

TABLE 1

| | | L | a | b |
|---|---|---|---|---|
| | Cultivar | (lightness/darkness) | (red/green) | (blue/yellow) |
| Skin | Bienville | 55.7 ± 1.6 | 21.6 ± 1.6 | 28.9 ± 1.3 |
| | | (2.6 YR)^{z} | (5.4) | (6.6) |
| | Beauregard | 53.7 ± 2.3 | 20.4 ± 1.8 | 24.2 ± 2.3 |
| | | (1.7 YR) | (5.2) | (6.1) |
| Flesh | Bienville | 66.4 ± 1.3 | 33.0 ± 1.2 | 41.1 ± 1.6 |
| | | (1.3) | (6.5) | (9.9) |
| | Beauregard | 69.4 ± 1.0 | 30.1 ± 1.9 | 38.8 ± 2.1 |
| | | (1.7 YR) | (6.8) | (9.3) |

^{z}Data in parentheses represent Munsell ® color equivalents for hue (L), value (a), and chroma (b).

### EXAMPLE 1

Tests Conducted

To confirm that Bienville was in fact a new variety, controlled tests (e.g., pathogen responses and yield) were conducted at the Louisiana Agricultural Experiment Station in Baton Rouge, La. As an ancestor of Bienville, Beauregard was used for comparison tests. Diseases that commonly affect the growth of sweet potatoes were used to test for pathogen responses in both varieties. Scions of Bienville and Beauregard reacted similarly to most diseases evaluated in the controlled tests. Bienville was resistant to soil rot caused by Streptomyces ipomoeae (Person & W. J. Martin) Waksman & Henrici. In severely infested fields, yield of Bienville was unaffected, and storage roots had few lesions. While yield of Beauregard was also unaffected, storage roots often had lesions. Bienville and Beauregard exhibited similar resistance to Fusarium wilt or stem rot caused by Fusarium oxysporum Schlect. f. sp. batatas (Wollenw.) Snyd. & Hans.

Nematode reproduction was measured in greenhouse tests. Bienville exhibited higher resistant to southern root-knot nematode, Meloidogyne incognita (Kofoid & White 1919) Chitwood 1949 races 1 and 3, than Beauregard. Bienville and Beauregard were both resistant to the development of internal cork, a disease presumably caused by a virus (unknown), but some storage roots developed an unusual discoloration of the vascular ring at the proximal end. Bienville and Beauregard exhibited similar resistance to Fusarium root rot caused by Fusarium solani (Sacc.) Mart. emend. Snyd. & Hans. At harvest, Bienville was more resistant to bacterial soft rot caused by Erwinia chrysanthemi Burkholder, McFadden & Dimock, than Beauregard. However, Bienville exhibited susceptibility to bacterial soft rot comparable to that of Beauregard after storage for three to five months. Bienville and Beauregard were both resistant to Rhizopus soft rot caused by Rhizopus stolonifer (Ehr. ex. Fr.) Lind. Bienville and Beauregard exhibited a similar incidence of circular spot caused by Sclerotium rolfsii Sacc.

Bienville did not appear to show any novel insect resistance. Bienville and Beauregard showed similar levels of susceptibility to important insect pests, notably the banded cucumber beetle, Diabrotica balteata LeConte, white grub, Plectris aliena Chapin or Phyllophaga spp., and the sweetpotato weevil, Cylas formicarius (fab.).

To determine yield, complete-block trials using four replications of Bienville and Beauregard each were conducted at two different Louisiana Agricultural Experiment Station locations, the Burden Research Plantation in Baton Rouge, La. in 2000, and the Sweet Potato Research Station in Chase, La. in 1997. Bienville and Beauregard were both transplanted in randomized complete-block trials at 31, 36, and 41 cm spacings in Loring silt loam soil at the Burden Research Plantation, and at 31 cm spacings in Gilbert silt loam soil at the Sweet Potato Research Station. Each block/plot was fertilized with 250 pounds per acre of nitrogen, P₂O₅, and K₂O (about 250 pounds per acre of 13% N, 13% P₂O₅, and 13% K₂O, 13-13-13 mixed fertilizer). Bienville was compared to Beauregard at early and middle transplanting dates at each location beginning around May. Average yields were measured for the following grades of roots: U.S. #1 (51-89 mm in diameter, 76-229 mm long); Canner (25-51 mm in diameter, 51-178 mm long); and Jumbo (larger than U.S. #1 in diameter, length or both, and without objectionable defects).

Early transplanting date trials were conducted at the Burden Research Plantation. Bienville and Beauregard were transplanted on June 13 and harvested on October 11 (120 days after planting). Average yields, measured as Mg·ha⁻¹, are shown in Table 2.

TABLE 2

| Selection | | | | |
|---|---|---|---|---|
| (spacing, in cm) | US#1 | Canners | Jumbos | TMY^{‡} |
| Bienville (41) | 16.3b^{†} | 8.0a | 0.2c | 24.4b |
| Bienville (36) | 22.7a | 7.0ab | 1.9bc | 31.5a |
| Bienville (31) | 21.4ab | 7.3ab | 1.1bc | 29.8ab |
| Beauregard (41) | 22.4a | 5.3b | 4.0ab | 31.7a |
| Beauregard (36) | 22.8a | 6.4ab | 5.6a | 34.8a |
| Beauregard (31) | 25.1a | 6.8ab | 4.2ab | 36.0a |
| Least Significant | 5.6 | 2.1 | 3.0 | 6.3 |
| Difference | | | | |
| LSD (P < 0.05) | | | | |

^{†}Average yields of varieties followed by a common letter do not differ significantly (P < 0.05) according to Duncan's Multiple Range Test.

Middle transplanting date trials were also conducted at the Burden Research Plantation. Bienville and Beauregard were transplanted on June 22 and harvested on October 27 (127 days after planting). Average yields (Mg·ha⁻¹) of Bienville and Beauregard are shown in Table 3.

TABLE 3

| Selection | | | | |
|---|---|---|---|---|
| (Spacing, in cm) | US#1 | Canners | Jumbos | TMY^{‡} |
| Bienville (41) | 17.4a^{†} | 5.9ab | 5.7a | 29.0ab |
| Bienville (36) | 16.7a | 4.6ab | 3.6a | 24.9ab |
| Bienville (31) | 14.4a | 5.7ab | 0.9a | 21.0ab |
| Beauregard (41) | 7.6a | 4.7ab | 4.6a | 16.9b |
| Beauregard (36) | 14.2a | 3.3b | 4.1a | 21.6ab |
| Beauregard (31) | 17.1a | 13.4a | 7.9a | 38.5a |
| Least Significant | 7.5 | 7.0 | 6.1 | 15.5 |
| Difference | | | | |
| LSD (P < 0.05) | | | | |

^{†}Average yields of varieties followed by a common letter do not differ significantly (P < 0.05) according to Duncan's Multiple Range Test.

Early transplanting date trials were also conducted at the Sweet Potato Research Station. Bienville was transplanted on May 16 and harvested on September 02 (109 days after planting). (Beauregard was not included in this transplanting trial.) Average yields (Mg·ha⁻¹) by grade are shown in Table 4.

TABLE 4

| Selection | | | | |
|---|---|---|---|---|
| (spacing, in cm) | US#1 | Canners | Jumbos | TMY^{‡} |
| Bienville (31) | 25.8a | 15.2a | 4.8a | 45.7a |
| Beauregard (31) | 25.8a | 11.5a | 6.4a | 45.6a |
| Least Significant | 6.2 | 6.0 | 4.9 | 5.8 |
| Difference | | | | |
| LSD (P < 0.05) | | | | |

^{†}Average yields of varieties followed by a common letter do not differ significantly (P < 0.05) according to Duncan's Multiple Range Test.
TMY^{‡} = total marketable yield

Middle transplanting date trials were also conducted at the Sweet Potato Research Station. Bienville and Beauregard were transplanted on June 13, and harvested on October 7 (116 days after planting) using a 31 cm spacing. Average yields (Mg·ha⁻¹) by grade are shown in Table 5.

TABLE 5

| Selection | | | | |
|---|---|---|---|---|
| (spacing, in cm) | US#1 | Canners | Jumbos | TMY^{‡} |
| Bienville (31) | 26.4b | 14.3a | 2.5a | 43.2b |
| Beauregard (31) | 35.9a | 12.6a | 8.1a | 56.5a |
| Least Significant | 7.9 | 4.1 | 6.4 | 9.3 |
| Difference | | | | |
| LSD (P < 0.05) | | | | |

^{†}Average yields of varieties followed by a common letter do not differ significantly (P < 0.05) according to Duncan's Multiple Range Test.

As shown in Tables 2-5, Bienville produced yields comparable to those of Beauregard at early transplanting dates (95% of Beauregard for U.S. #1 grade; 89% of Beauregard for total marketable yield). Spacing had no significant effect on yield. At later planting dates, Bienville had yields slightly less than those of Beauregard at the Sweetpotato Research Station, but were still competitive. Replicated plots on sweet potato production farms have not shown any predisposition of Bienville to low yield in late plantings.

Bienville produces plants (sprouts) one week later than Beauregard, in a quantity of approximately 30 to 40% less than Beauregard. Days to harvest are similar to Beauregard. Yields for both total and number one grade roots are typically similar to Beauregard; however, yield may sometimes be slightly less than that of Beauregard.

Bienville's primary expected use is as a commercial variety produced on land infested with southern root-knot nematode, soil rot, or both. Beauregard produces inferior quality roots in such soils.

We claim:

1. A sweet potato plant substantially as described and illustrated in the specification herein.
